(19) United States
(12) Patent Application Publication
(10) Pub. No.: US 2025/0134401 A1
(43) Pub. Date: May 1, 2025

(54) SPHYGMOMANOMETER, BLOOD PRESSURE MEASUREMENT METHOD, AND KOROTKOFF SOUND DETECTION DEVICE

(71) Applicant: OMRON HEALTHCARE CO., LTD., Muko-shi (JP)

(72) Inventors: Wataru TSUNODA, Kyoto (JP); Terumasa Naito, Kyoto (JP); Yukiya Sawanoi, Kyoto (JP)

(73) Assignee: OMRON HEALTHCARE CO., LTD., Muko-shi (JP)

(21) Appl. No.: 19/008,850

(22) Filed: Jan. 3, 2025

Related U.S. Application Data
(63) Continuation of application No. PCT/JP2023/028940, filed on Aug. 8, 2023.

(30) Foreign Application Priority Data
Oct. 25, 2022 (JP) 2022-170781

(51) Int. Cl. A61B 5/0225; A61B 5/022

(52) U.S. Cl. CPC A61B 5/0225; A61B 5/02208

(57) ABSTRACT

The present invention includes a diaphragm disposed to receive, on one surface, a pressure of a first air in an air pipe as a part of a pipe wall of the air pipe coupled to a cuff in a fluid flowable manner. The diaphragm blocks the pressure of the first air in the air pipe in a pressurization process or a depressurization process of the cuff, meanwhile transmits, through this diaphragm, a sound in a frequency band of a Korotkoff sound among sounds traveled through the first air in the air pipe from a measurement target site. A chamber disposed including the diaphragm as a part of a peripheral wall on a side of an other surface of the diaphragm is included. A sound detection device provided to face a second air in the chamber is included.

### CROSS-REFERENCE TO RELATED APPLICATIONS

This is a continuation application of International Application No. PCT/JP2023/028940, with an International filing date of Aug. 8, 2023, which claims priority of Japanese Patent Application No. 2022-170781 filed on Oct. 25, 2022, the entire content of which is hereby incorporated by reference.

### TECHNICAL FIELD

This invention relates to a sphygmomanometer and a blood pressure measurement method, and more particularly to a sphygmomanometer and a blood pressure measurement method for measuring a blood pressure based on a Korotkoff sound generated by a measurement target site. This invention relates to a Korotkoff sound detection device that is included in such a sphygmomanometer and extracts a Korotkoff sound from sounds generated by a measurement target site.

### BACKGROUND ART

As this type of Korotkoff sound detection device (and sphygmomanometer), as disclosed in FIG. 2 of Patent Document 1 (JP-A-S58-180132), for example, there is a known device including a vibration film (metal film) that vibrates by a sound collected from a cuff belt wound around an upper arm of a human body and pressurized and has a mechanical resonance point in a frequency band where a Korotkoff sound exists, and a means for converting the mechanical vibration of this vibration film into an electric signal. This configuration is understood as a condenser microphone. The document describes that it is possible to extract only a Korotkoff sound by removing noise components other than the Korotkoff sound by the mechanical resonance characteristics of the vibration film.

### SUMMARY OF THE INVENTION

However, the sound (in a broad sense, defined as a wave propagating in an elastic medium such as air) generated by a measurement target site includes, in addition to a Korotkoff sound (frequency band; about 20 Hz to 500 Hz), and a pressure pulse wave (frequency band; about several tens of Hz), which is pulse wave vibration in an artery through the measurement target site. Both frequency bands overlap each other, and the amplitude of the pressure pulse wave is larger than the amplitude of the Korotkoff sound. Therefore, in practice, the configuration described in Patent Document 1 has a problem of difficulty in extracting only a Korotkoff sound from sounds generated by the measurement target site.

Therefore, an object of this invention is to provide a sphygmomanometer and a blood pressure measurement method for measuring a blood pressure based on a Korotkoff sound generated by a measurement target site, the sphygmomanometer and the blood pressure measurement method being able to extract, with a good signal-to-noise ratio (S/N ratio), a Korotkoff sound from sounds generated by the measurement target site, and thus enhance an accuracy of blood pressure measurement. An object of this invention is to provide a Korotkoff sound detection device that is included in such a sphygmomanometer and can extract a Korotkoff sound with a good S/N ratio from sounds generated by a measurement target site.

In order to achieve the object, a sphygmomanometer of the present disclosure is a sphygmomanometer that measures a blood pressure based on a Korotkoff sound generated by a measurement target site, the sphygmomanometer comprising:
a cuff for pressure configured to be worn to the measurement target site;
a pump;
an air pipe coupling the cuff and the pump in a fluid flowable manner;
a pressure control unit that supplies and pressurizes a first air to the cuff through the air pipe by the pump in order to press the measurement target site, or discharges and depressurizes the first air from the cuff through the air pipe;
a diaphragm disposed so as to receive, on one surface, a pressure of the first air in the air pipe as a part of a pipe wall of the air pipe or in connection with the pipe wall, the diaphragm being configured to block the pressure of the first air in the air pipe in a pressurization process or a depressurization process of the cuff by the pressure control unit, meanwhile transmit, through this diaphragm, a sound in a frequency band of the Korotkoff sound among sounds traveled through the first air in the air pipe from the measurement target site;
a chamber disposed including the diaphragm as a part of a peripheral wall on a side of an other surface opposite to the one surface with respect to the diaphragm, such that the sound transmitted through the diaphragm travels through a second air contained in the chamber;
a sound detection device provided to face the second air in the chamber in a part other than the diaphragm in the peripheral wall of the chamber, the sound detection device receiving, through the second air in the chamber, the sound transmitted through the diaphragm, and converting the sound into an electric signal; and
a blood pressure calculation unit that calculates the blood pressure of the measurement target site based on the electric signal.

Here, the “one surface” and the “other surface” of the diaphragm refer to both surfaces with a spreading of the diaphragm.

That the diaphragm is disposed “in connection with a pipe wall” of the air pipe includes, for example, a manner of being disposed in connection with a pipe wall of another pipe branched from the air pipe.

In another aspect, a blood pressure measurement method of the present disclosure is a blood pressure measurement method for measuring the blood pressure based on the Korotkoff sound generated by the measurement target site by the sphygmomanometer according to claim 1, the blood pressure measurement method comprising:
in the state where the cuff for pressure is worn to the measurement target site,
supplying and pressurizing, by the pressure control unit, the first air to the cuff through the air pipe by the pump in order to press the measurement target site, or discharging and depressurizing the first air from the cuff through the air pipe;
receiving, on the one surface by the diaphragm, in the pressurization process or the depressurization process of the cuff by the pressure control unit, the pressure of the first air in the air pipe to block the pressure of the first air in the air pipe, and transmitting, through this diaphragm, the sound in the frequency band of the Korotkoff sound among the sounds traveled through the first air in the air pipe from the measurement target site, such that the sound transmitted through the diaphragm travels through the second air contained in the chamber;
receiving, by the sound detection device, through the second air inside the chamber, the sound transmitted through the diaphragm, and converting the sound into the electric signal; and
calculating, by the blood pressure calculation unit, the blood pressure of the measurement target site based on the electric signal.

In another aspect, a Korotkoff sound detection device of the present disclosure is a Korotkoff sound detection device that is included in the sphygmomanometer according to claim 1 and extracts a Korotkoff sound from sounds generated by the measurement target site, the Korotkoff sound detection device comprising:
the diaphragm disposed so as to receive, on one surface, the pressure of the first air in the air pipe as the part of the pipe wall of the air pipe or in connection with the pipe wall, the diaphragm being configured to block the pressure of the first air in the air pipe in the pressurization process or the depressurization process of the cuff by the pressure control unit, meanwhile transmit, through this diaphragm, the sound in the frequency band of the Korotkoff sound among sounds traveled through the first air in the air pipe from the measurement target site;
the chamber disposed including the diaphragm as the part of the peripheral wall on the side of the other surface opposite to the one surface with respect to the diaphragm, such that the sound transmitted through the diaphragm travels through the second air contained in the chamber; and
the sound detection device provided to face the second air in the chamber in the part other than the diaphragm in the peripheral wall of the chamber, the sound detection device receiving, through the second air in the chamber, the sound transmitted through the diaphragm, and converting the sound into the electric signal.

### BRIEF DESCRIPTION OF THE DRAWINGS

FIG. 1 is a view showing a block configuration of a sphygmomanometer of an embodiment of this invention.

FIG. 2A is a perspective view showing a cross-sectional structure of a Korotkoff sound detection device included in the sphygmomanometer. FIG. 2B is a view showing a model for setting a resonant frequency of a chamber included in the Korotkoff sound detection device.

FIGS. 3A, 3B, and 3C are views showing various examples of a narrow tube forming a hole for pressure relaxation that communicates between an inside and an outside of the chamber in a fluid flowable manner. FIG. 3D is a view showing a verification result of noise reduction in the narrow tube shown in FIG. 3C by accommodating a sound insulation material in the narrow tube.

FIG. 4 is a view showing a state in which a cuff for pressure of the sphygmomanometer is worn to an upper arm as a measurement target site.

FIG. 5 is a view showing a flow of blood pressure measurement by the sphygmomanometer.

FIGS. 6A and 6B are views schematically describing the operation of the Korotkoff sound detection device during blood pressure measurement.

FIG. 7 is a view showing an example of a sound signal output by the Korotkoff sound detection device in the sphygmomanometer (embodiment).

FIG. 8 is a view showing an example of a sound signal output by a Korotkoff sound detection device of a comparative example.

FIGS. 9A and 9B are views each showing a modification of the hole for pressure relaxation.

### DETAILED DESCRIPTION

An embodiment of this invention will be described below in detail with reference to the drawings.

### Schematic Configuration of Sphygmomanometer

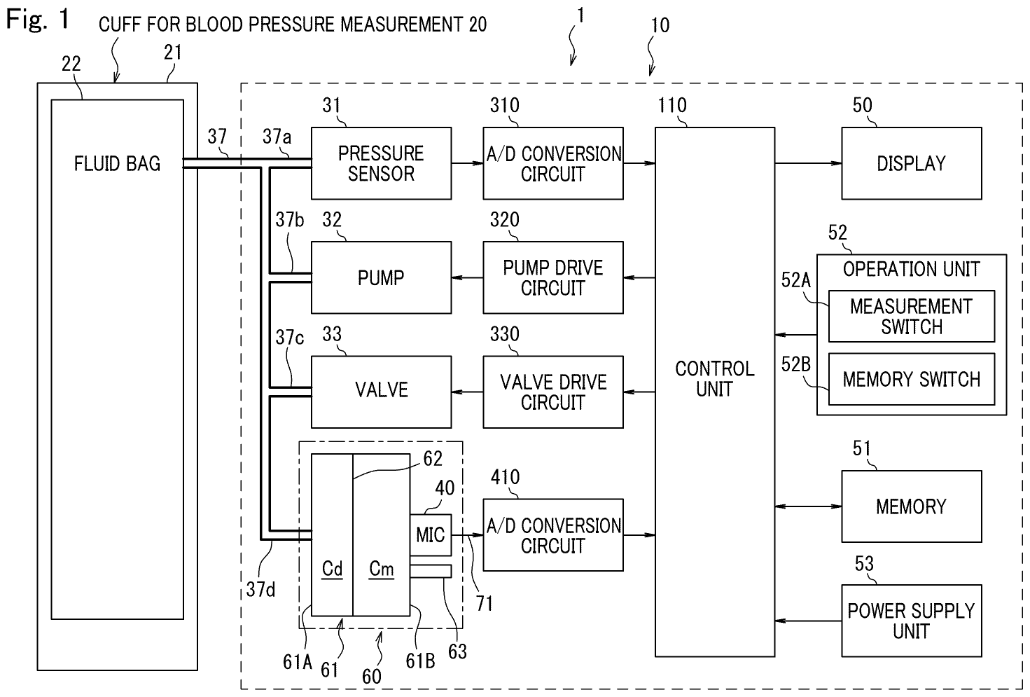

FIG. 1 is a view showing a block configuration of a sphygmomanometer 1 of an embodiment of this invention. This sphygmomanometer 1 roughly includes a cuff 20 for pressing (hereinafter, simply called a “cuff”) worn around the measurement target site (in this example, an upper arm), and a main body 10 connected to this cuff 20 via an air pipe 37 in a fluid flowable manner.

The cuff 20 includes a bag body 21, and this bag body 21 is configured by facing an outer fabric and an inner fabric each having an elongated band shape to each other and sewing (or welding) peripheral edge portions thereof. This bag body 21 internally accommodates a fluid bag 22 for pressing the measurement target site.

The main body 10 is mounted with a control unit 110, a display 50, an operation unit 52, a memory 51, a power supply unit 53, a pressure sensor 31, a pump 32, a valve 33, and a Korotkoff sound detection device 60 for extracting a Korotkoff sound from sounds generated by the measurement target site. Furthermore, the main body 10 is mounted with an A/D conversion circuit 310 that converts an analog output from the pressure sensor 31 into a digital signal, a pump drive circuit 320 that drives the pump 32, a valve drive circuit 330 that drives the valve 33, and an A/D conversion circuit 410 that converts an analog output from the Korotkoff sound detection device 60 into a digital signal. Air pipes 37a, 37b, 37c, and 37d are connected to the pressure sensor 31, the pump 32, the valve 33, and the Korotkoff sound detection device 60, respectively, in a fluid flowable manner. Those air pipes 37a, 37b, 37c, and 37d join to one air pipe 37 in the main body 10, and this air pipe 37 is connected to the fluid bag 22 in the cuff 20 in a fluid flowable manner. Hereinafter, the air pipes 37a, 37b, 37c, and 37d are collectively called the air pipe 37 as appropriate. The output of the Korotkoff sound detection device 60 is transmitted to the A/D conversion circuit 410 by wiring 71 as a sound signal Ks that is an electric signal.

In this example, the display 50 includes a display, an indicator and the like, and displays predetermined information (e.g., a blood pressure measurement result or the like) in accordance with a control signal from the control unit 110.

The operation unit 52 inputs, to the control unit 110, an operation signal in response to a user's instruction. In this example, the operation unit 52 includes a measurement switch 52A for receiving an instruction to start/stop a measurement of blood pressure and a memory switch 52B for receiving an instruction to call data of a measurement result of a blood pressure value stored in the memory 51.

The memory 51 stores, as a storage unit, data of a program for controlling the sphygmomanometer 1, setting data for setting various functions of the sphygmomanometer 1, data of the measurement result of the blood pressure value, and the like. The memory 51 is used as a work memory or the like when a program is executed.

The control unit 110 includes a central processing unit (CPU) and controls the entire operation of this sphygmomanometer 1. Specifically, the control unit 110 serves as a pressure control unit in accordance with a program for controlling the sphygmomanometer 1 stored in the memory 51, and performs control of driving the pump 32 and the valve 33 in response to an operation signal from the operation unit 52. The control unit 110 serves as a threshold setting unit and a blood pressure calculation unit, calculates a blood pressure value of the measurement target site based on the sound signal Ks output from the Korotkoff sound detection device 60, and controls the display 50 and the memory 51. A specific method of blood pressure measurement will be described later.

The power supply unit 53 supplies power to each unit of the control unit 110, the pressure sensor 31, the pump 32, the valve 33, the display 50, the memory 51, the A/D conversion circuits 310 and 410, the pump drive circuit 320, the valve drive circuit 330, and a microphone 40 described later. The microphone 40 is supplied with power through the wiring 71.

The pressure sensor 31 is a piezoresistive pressure sensor in this example, and upon receiving a pressure (this is called “cuff pressure Pc”) of the cuff 20 (in this example, the fluid bag 22) through the air pipe 37, and outputs an electric signal value based on a change in electric resistance due to a piezoresistive effect to the control unit 110 through the A/D conversion circuit 310. The control unit 110 detects the cuff pressure Pc in response to the electric signal value from the pressure sensor 31.

The pump 32 supplies an air (first air) to the fluid bag 22 through the air pipe 37 in order to pressurize the cuff pressure Pc. The valve 33 is opened and closed to control the cuff pressure Pc by discharging or enclosing the air in the fluid bag 22 through the air pipe 37. The pump drive circuit 320 drives the pump 32 based on a control signal given from the control unit 110. The valve drive circuit 330 opens and closes the valve 33 based on a control signal given from the control unit 110.

In this example, the Korotkoff sound detection device 60 generally includes a case 61 having a substantially short cylindrical shape, a diaphragm 62 provided across an inside of the case 61, the microphone 40 as a sound detection device, and a narrow tube 63 forming a hole for pressure relaxation.

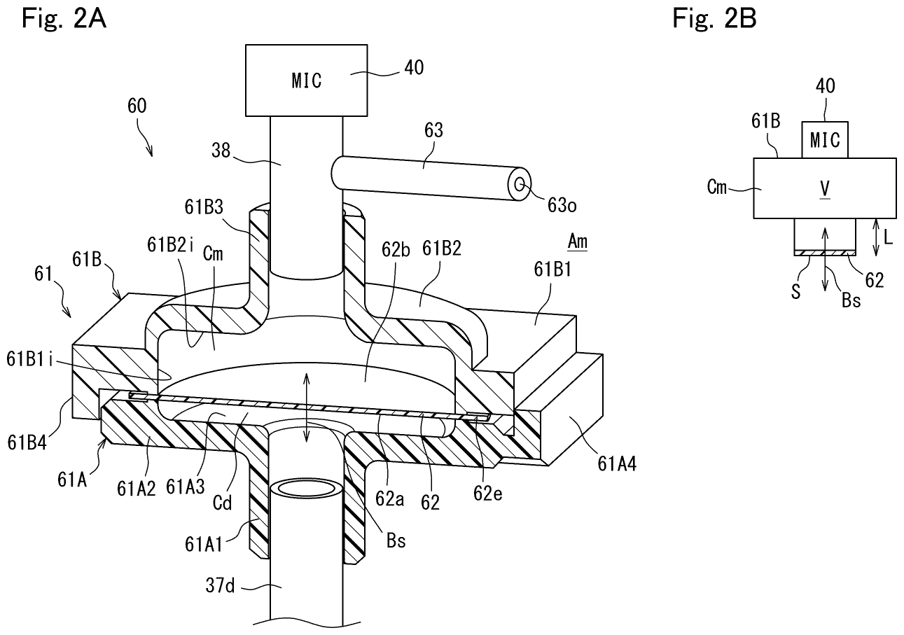

FIG. 2A shows an example of a cross-sectional structure of the Korotkoff sound detection device 60. This Korotkoff sound detection device 60 includes the case 61 including a lower case 61A and an upper case 61B. FIG. 2A shows a longitudinal cross section of the case 61 cut in half with a vertical plane for easy understanding. Note that the terms “lower”, “upper”, “vertical”, and “horizontal” described later are used for convenience of description, and the case 61 can be integrally disposed in any orientation in the main body 10 of the sphygmomanometer 1.

The lower case 61A includes a cylindrical portion 61A1 fitted around an air pipe 37d, a plate portion 61A2 having a substantially rectangular plate-like outer shape horizontally extending from an upper end of the cylindrical portion 61A1, a recess portion 61A3 provided as a circular recess having a flat bottom on an upper surface of this plate portion 61A2, and an edge portion 61A4 bent from an end side (right side in FIG. 2A) of the plate portion 61A2 and extending upward. The cylindrical portion 61A1 is airtightly fitted around the air pipe 37d. A space Cd made on the plate portion 61A2 by the recess portion 61A3 communicates with the air pipe 37d (accordingly, the air pipe 37) via the cylindrical portion 61A1 in a fluid flowable manner. In this example, the horizontal dimension of the plate portion 61A2 is set to about 50 mm.

The upper case 61B includes a plate portion 61B1 having a substantially rectangular shape that extends substantially in parallel facing the plate portion 61A2 of the lower case 61A, a dome portion 61B2 that protrudes upward in a circular dome shape from the plate portion 61B1, a cylindrical portion 61B3 extending upward from a substantial center of the dome portion 61B2, and an edge portion 61B4 bent from an end side (left side in FIG. 2A) of the plate portion 61B1 and extending downward. In this example, the horizontal dimension of the plate portion 61B1 is set to about 50 mm similarly to that of the plate portion 61A2 of the lower case 61A. The horizontal position and dimension of the dome portion 61B2 substantially coincide with the horizontal position and dimension of the recess portion 61A3 of the lower case 61A. In this example, an air pipe 38 is airtightly fitted and inserted into the cylindrical portion 61B3. The microphone 40 is airtightly attached to an upper end of the air pipe 38. In this example, the narrow tube 63 is attached in the middle of the air pipe 38. A space Cm (forming a chamber described later) made by an inner peripheral edge 61B1i of the plate portion 61B1 and an inner edge 61B2i of the dome portion 61B2 communicates with the microphone 40 in a fluid flowable manner via the cylindrical portion 61B3 and the air pipe 38.

The diaphragm 62 having a substantially circular film shape is provided between the plate portion 61A2 of the lower case 61A and the plate portion 61B1 of the upper case 61B transversely so as to partition the space Cd and the space Cm. In this example, a peripheral edge portion 62e of the diaphragm 62 is held between the plate portion 61A2 of the lower case 61A and the plate portion 61B1 of the upper case 61B. Thereby, a part of the diaphragm 62 other than the peripheral edge portion 62e can vibrate up and down as indicated by arrow Bs in FIG. 2A. The peripheral edge portion 62e of the diaphragm 62 may be bonded to the plate portion 61A2 of the lower case 61A and/or the plate portion 61B1 of the upper case 61B with an adhesive. In this example, the diaphragm 62 includes a polyurethane sheet (0.3 mm in thickness) as a synthetic resin. In this example, the effective radius (substantially equal to the horizontal radii of the spaces Cd and Cm) R of the diaphragm 62 is set to R=16.5 mm. Therefore, this diaphragm 62 is lighter in weight than that including metal, and is easy to process at the manufacturing stage. In this example, the natural frequency of the diaphragm 62 is set to match a frequency band (about 20 Hz to 500 Hz) of the Korotkoff sound. Therefore, the diaphragm 62 can selectively transmit a sound in the frequency band of Korotkoff sound.

The edge portion 61A4 of the lower case 61A and the edge portion 61B4 of the upper case 61B are provided for convenience of aligning the lower case 61A and the upper case 61B with each other in a horizontal plane when the lower case 61A and the upper case 61B are assembled. Thanks to these edge portions 61A4 and 61B4, the recess portion 61A3 of the lower case 61A and the dome portion 61B2 of the upper case 61B can be concentrically and easily aligned.

In this example, an upper surface 62b of the diaphragm 62, the inner peripheral edge 61B1i of the plate portion 61B1, an inner surface 61B2i of the dome portion 61B2, the cylindrical portion 61B3, and the air pipe 38 constitute a chamber (for simplicity, represented by the same reference sign as the space Cm). In this example, the chamber Cm is set to have a resonant frequency that matches the frequency band (about 20 Hz to 500 Hz) of Korotkoff sound.

Specifically, FIG. 2B shows a model of Helmholtz resonance in a case of a configuration in which the cylindrical portion 61B3 of the upper case 61B and the air pipe 38 are omitted and the chamber Cm communicates with the microphone 40 in a fluid flow manner (note that in that case, as shown in FIG. 1, the narrow tube 63 may be directly attached to the dome portion 61B2 of the upper case 61B or may be formed integrally with the dome portion 61B2). In FIG. 2B, S represents an effective area (area of actually vibrating portion, in unit m²) of the diaphragm 62, V represents an internal volume (in unit m³) of the chamber Cm, and L represents an equivalent neck length (in unit m). In this case, a resonant frequency f of the chamber Cm is calculated with the theory of Helmholtz resonance by an equation (Eq. 1).

$$f = ( c / 2 π ) {( S / VL )}^{1 / 2} \qquad ( Eq . 1 )$$

Here, c represents a sound speed, and c≈340 m/sec. In this example, the resonant frequency f of the chamber Cm is set to match the frequency band (about 20Hz to 500 Hz) of the Korotkoff sound based on the equation (Eq. 1).

Therefore, the chamber Cm can selectively amplify the sound in the frequency band of the Korotkoff sound among the sounds transmitted through the diaphragm 62.

The microphone 40 receives, through an air (second air) contained in the chamber Cm, the sound transmitted through the diaphragm 62, and converts the sound into the sound signal Ks, which is an electric signal. The sound signal Ks mainly includes a component representing a Korotkoff sound. The sound signal Ks is transmitted to the control unit 110 via the wiring 71 and the A/D conversion circuit 410 as an output of the Korotkoff sound detection device 60.

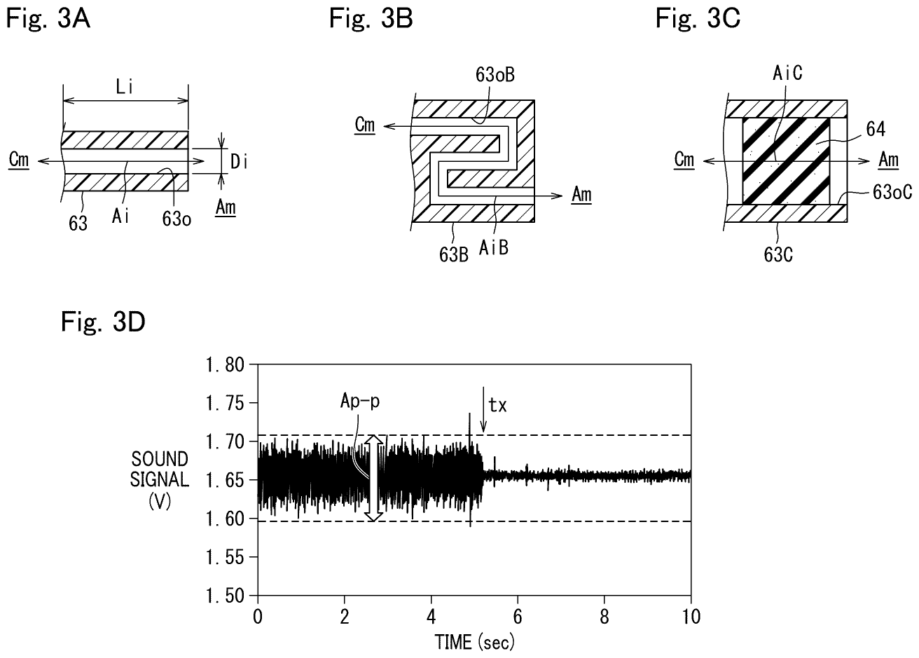

In this example, the narrow tube 63 forming the hole for pressure relaxation has a cylindrical outer shape. As shown in FIG. 3A, the narrow tube 63 is provided with a hole 63o for pressure relaxation that communicates between an inside and an outside of the chamber Cm in a fluid flowable manner. In this example, the hole 63o is in a form of an elongated duct extending straight. In this example, an axial dimension Li of the narrow tube 63 is set to about several mm to several cm. An inner diameter Di of the hole 63o is set to about 0.1 mm to several mm.

When the pressure of the air in the air pipe 37 (and the space Cd) gradually changes and the diaphragm 62 bends in the pressurization process or the depressurization process of the cuff 20, and thereby the pressure of the air in the chamber Cm is about to change, the hole 63o serves to communicate air between the inside and the outside of the chamber Cm as indicated by arrow Ai in FIG. 3A to restrain the pressure of the air in the chamber Cm from changing from an atmospheric pressure (ambient pressure) Am. Therefore, even if the pressure of the air in the air pipe 37 (and the space Cd) changes, it is possible to prevent the sensitivity, resolution, durability, and reliability of the microphone 40 from being adversely affected by the pressure change (load).

Here, the hole 63o for pressure relaxation is in a form of an elongated duct. Therefore, noise sounds are less likely to enter the chamber Cm from the outside of the chamber Cm through the hole 63o as compared with a case where the hole 63o for pressure relaxation is, for example, a wide opening (not shown). Therefore, it is possible to prevent the S/N ratio of the Korotkoff sound from decreasing due to the hole for pressure relaxation. Note that in the example of FIG. 3A, the hole 63o is straight, but the present invention is not limited to this. For example, in a narrow tube 63B shown in FIG. 3B, a hole 63oB for pressure relaxation provided inside thereof is in a form of an elongated duct that reciprocates in a zigzag manner. Also in this case, the hole 63oB serves to communicate air between the inside and the outside of the chamber Cm as indicated by arrow AiB to restrain the pressure of the air in the chamber Cm from changing from the atmospheric pressure Am. Therefore, even if the pressure of the air in the air pipe 37 (and the space Cd) changes, it is possible to prevent the sensitivity, resolution, durability, reliability, and the like of the microphone 40 from being adversely affected by the pressure change (load). Moreover, as compared with the example of FIG. 3A, noise sounds are less likely to enter the chamber Cm from the outside of the chamber Cm through the hole 63oB. Therefore, it is possible to further prevent the S/N ratio of the Korotkoff sound from decreasing due to the hole for pressure relaxation.

In a narrow tube 63C shown in FIG. 3C, a hole 63oC for pressure relaxation provided inside thereof is in a form of an elongated duct extending straight. However, as a sound insulation material having air permeability and sound insulation properties, polyurethane foam 64, which is a porous material in this example, is accommodated inside the hole 63oC. This polyurethane foam 64 has air permeability as indicated by arrow AiC. Therefore, the function of the hole 63oC that restrains the pressure of the air in the chamber Cm from changing from the atmospheric pressure Am is not lost due to the presence of the polyurethane foam 64. Since the polyurethane foam 64 has sound insulation properties, noise sounds are less likely to enter the chamber Cm from the outside of the chamber Cm through the hole 63oC compared with the case where only air exists inside the hole 63oC. Therefore, it is possible to further prevent the S/N ratio of the Korotkoff sound from decreasing due to the hole 63oC. For example, FIG. 3D shows a result of verifying what degree noise sound in the chamber Cm is reduced by accommodating the polyurethane foam 64 inside the hole 63oC when the axial dimension Li of the narrow tube 63C is 2 mm and the inner diameter Di of the hole 63oC is about 0.2 mm. In this example, before the polyurethane foam 64 is accommodated inside the hole 63oC (before time tx), a background noise level (peak-to-peak) Ap-p in the chamber Cm was about 0.11 V. In contrast, after the polyurethane foam 64 is accommodated inside the hole 63oC (after time tx), the background noise level Ap-p in the chamber Cm was reduced to about 0.02 V. In this manner, it was verified to be possible to effectively reduce the noise sound in the chamber Cm by the existence of the polyurethane foam 64.

### Blood Pressure Measurement Method

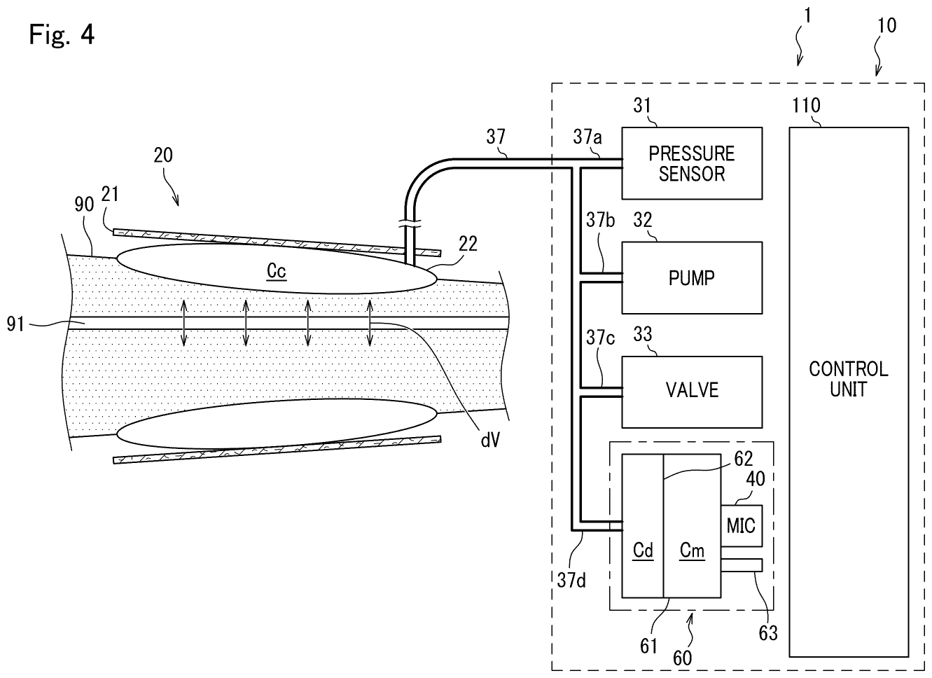

At the time of blood pressure measurement, as shown in FIG. 4, the cuff 20 is worn around a measurement target site (in this example, the upper arm) 90 of the user (note that in FIG. 4, the inner fabric is not shown for simplicity). It is assumed that an artery 91 passes through the measurement target site 90. The sound generated by the measurement target site 90 includes, in addition to a Korotkoff sound (frequency band; about 20 Hz to 500 Hz), and a pressure pulse wave (frequency band; about several tens of Hz) dV, which is pulse wave vibration in the artery 91 passing through the measurement target site 90. The sound generated by the measurement target site 90 travels from a space Cc made by the fluid bag 22 to (the space Cd of) the Korotkoff sound detection device 60 in the main body 10 through the air pipe 37.

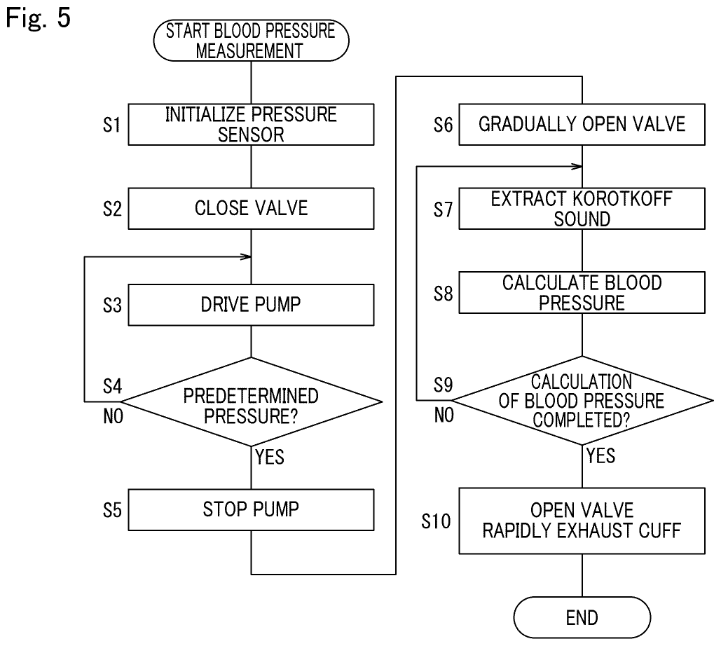

FIG. 5 shows an operation flow when the user performs blood pressure measurement with the sphygmomanometer 1.

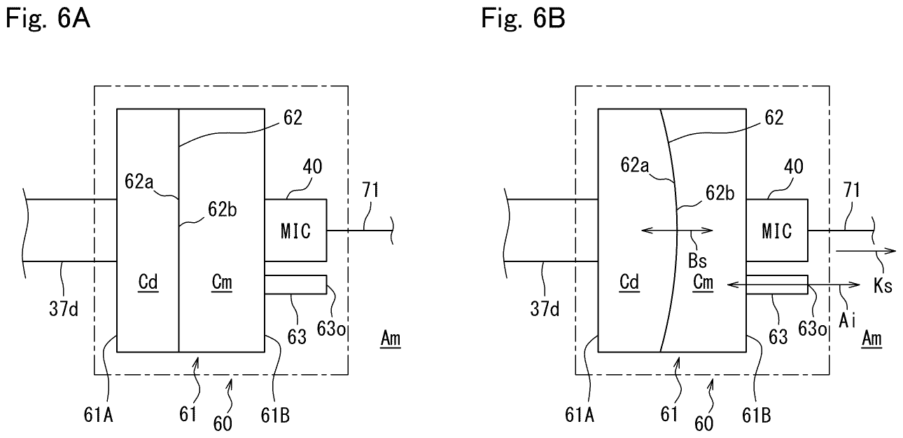

When the user instructs a start of measurement with the measurement switch 52A of the operation unit 52 provided on the main body 10 in a worn state where the cuff 20 is worn to the measurement target site, the control unit 110 performs initialization (step S1 in FIG. 5). Specifically, the control unit 110 initializes a processing memory area, and performs 0 mmHg adjustment (sets atmospheric pressure to 0 mmHg) of the pressure sensor 31 in a state where the pump 32 is turned off (stopped) and the valve 33 is opened. In this initial state, as shown in FIG. 6A, the diaphragm 62 of the Korotkoff sound detection device 60 is in a flat state.

Next, the control unit 110 serves as a pressure control unit, closes the valve 33 via the valve drive circuit 330 (step S2 in FIG. 5), and then turns on (drives) the pump 32 via the pump drive circuit 320 to start pressurization of the cuff 20 (fluid bag 22) (step S3). That is, the control unit 110 supplies the air (first air) as a fluid from the pump 32 to the fluid bag 22 in the cuff 20 through the air pipe 37. Along with this, the pressure sensor 31 receives the cuff pressure Pc through the air pipe 37. The control unit 110 controls the pressurization speed by the pump 32 based on the output of the pressure sensor 31.

In this pressurization process, as shown in FIG. 6B, the diaphragm 62 of the Korotkoff sound detection device 60 receives, on one surface (surface on the space Cd side) 62a, the pressure of the air in the air pipe 37 (in particular, the air pipe 37d) and bends convexly toward the other surface 62b. This blocks the pressure of the air in the air pipe 37. When the pressure of the air (second air) in the chamber Cm is about to change due to bending of the diaphragm 62, the hole 63o for pressure relaxation of the narrow tube 63 serves to communicate air between the inside and the outside of the chamber Cm as indicated by arrow Ai to restrain the pressure of the air in the chamber Cm from changing from the atmospheric pressure Am.

Next, in step S4 of FIG. 5, the control unit 110 determines whether or not the cuff pressure Pc has reached a predetermined value (predetermined pressure) based on the output of the pressure sensor 31. Here, the predetermined pressure may be set to be, for example, 180 mmHg so as to sufficiently exceed a blood pressure value assumed for the user, or may be set to be a blood pressure value of the user measured last time plus 40 mmHg. The control unit 110 continues pressurization until the cuff pressure Pc reaches the predetermined pressure, and stops (step S5) the pump 32 when the cuff pressure Pc reaches the predetermined pressure (YES in step S4). Subsequently, the control unit 110 gradually opens the valve 33 via the valve drive circuit 330 (step S6). This depressurizes the cuff pressure Pc at a substantially constant speed.

During this depressurization process, as shown in FIG. 6B, the diaphragm 62 receives the pressure of the air in the air pipe 37 on the one surface 62a, blocks the pressure of the air in the air pipe 37, and vibrates as indicated by arrow Bs to transmit the sound in the frequency band of the Korotkoff sound through the diaphragm 62. As a result, the sound transmitted through the diaphragm 62 travels through the air in the chamber Cm. The microphone 40 receives, via the air in the chamber Cm, the sound transmitted through the diaphragm 62, converts the sound into the sound signal Ks, which is an electric signal, and outputs the sound signal Ks via the wiring 71.

Here, since the diaphragm 62 blocks the pressure of the air in the air pipe 37, it is possible to reduce the influence of the pressure pulse wave dV, which is pulse wave vibration of the artery 91, from the sound generated by the measurement target site 90. Moreover, the microphone 40 receives, through the air in the chamber Cm, the sound transmitted through the diaphragm 62, in other words, receives it at a position spaced apart from the diaphragm 62 (directly receives the pressure pulse wave). Therefore, the influence of the pressure pulse wave dV can be further reduced. This can remove noises due to the pressure pulse wave dV from the sound generated by the measurement target site 90.

Since the diaphragm 62 is set to have a natural frequency matching the frequency band of the Korotkoff sound, it is possible to selectively transmit the sound in the frequency band of the Korotkoff sound among sounds traveled from the measurement target site 90 through the air in the air pipe 37 (and the space Cd). Furthermore, since the chamber Cm is set to have a resonant frequency matching the frequency band of the Korotkoff sound, it is possible to selectively amplify the sound in the frequency band of the Korotkoff sound among the sounds transmitted through the diaphragm 62. Therefore, it is possible to extract a Korotkoff sound with a good S/N ratio.

In this depressurization process, as shown in step S7 (Korotkoff sound extraction processing) of FIG. 5, the control unit 110 acquires, via the A/D conversion circuit 410, the sound signal Ks output from (the microphone 40 of) the Korotkoff sound detection device 60, and extracts a signal (called a “Korotkoff sound signal Kc”) representing the Korotkoff sound from the sound signal Ks.

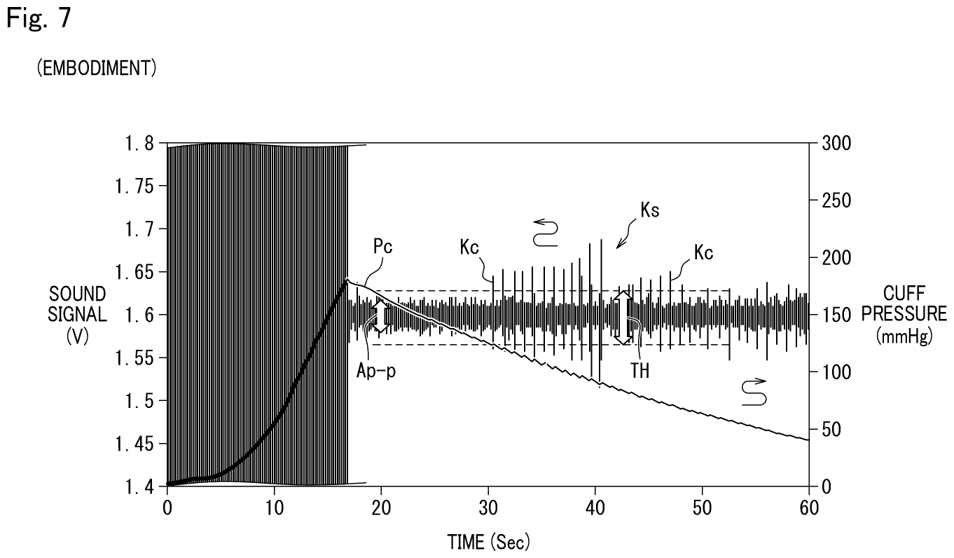

Specifically, FIG. 7 shows an example of the sound signal Ks output from the Korotkoff sound detection device 60. A mountain-shaped curve in FIG. 7 represents the cuff pressure Pc. In this example, the cuff pressure Pc reaches a predetermined pressure of 180 mmHg after about 17 seconds from the start of pressurization, and the depressurization process is started from that time point. In this example, the sound signal Ks includes a plurality of Korotkoff sound signals Kc in pulses that exceed the background noise level Ap-p (in this example, approximately 0.02 V). In this example, the control unit 110 serves as a threshold setting unit and sets a threshold TH (in this example, approximately 0.06 V) exceeding the background noise level Ap-p for the sound signal Ks. Then, the control unit 110 extracts, as the Korotkoff sound signal Kc, only a signal exceeding the threshold TH among the sound signals Ks. This can remove the background noise from the sound signal Ks. Therefore, the S/N ratio of the Korotkoff sound can be further improved. Along with this, under the control of the control unit 110, the memory 51 stores an amplitude of the Korotkoff sound signal Kc having been extracted and a time when the Korotkoff sound signal Kc is generated in association with each other, repeatedly.

Thereafter, in step S8 of FIG. 5, the control unit 110 serves as a blood pressure calculation unit, and calculates the blood pressure of the measurement target site based on the Korotkoff sound signal Kc stored in the memory 51. Specifically, in the depressurization process, the cuff pressure Pc at the time when the Korotkoff sound signal Kc appeared for the first time is determined as a systolic blood pressure SYS, and the cuff pressure Pc at the time when the Korotkoff sound signal Kc appeared for the last time is determined as a diastolic blood pressure DIA.

Upon calculating the blood pressure values (the systolic blood pressure SYS and the diastolic blood pressure DIA) in this manner (YES in step S9), the control unit 110 serves as a pressure control unit, and performs control of turning off the pump 32, opening the valve 33, and rapidly exhausting the air in the cuff 20 (fluid bag 22) (step S10). Thereafter, the control unit 110 performs control of displaying the calculated blood pressure values on the display 50 and saving the blood pressure values in the memory 51.

As described above, according to this blood pressure measurement method, it is possible to extract a Korotkoff sound with a good S/N ratio from sounds generated by the measurement target site 90, and thus it is possible to enhance an accuracy of blood pressure measurement.

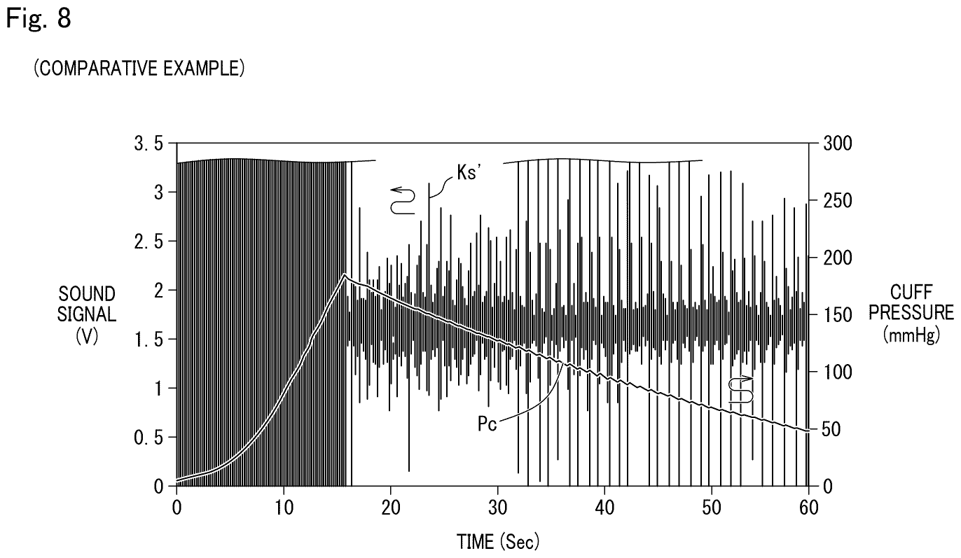

As a comparative example, the present inventor prepared a sphygmomanometer including a Korotkoff sound detection device along FIG. 2 of Patent Document 1 (JP-A-S58-180132). Note that parts of the sphygmomanometer other than the Korotkoff sound detection device have the same configurations as those of the sphygmomanometer 1 described above. FIG. 8 shows a sound signal Ks' output from the Korotkoff sound detection device. Also in this example of FIG. 8, the cuff pressure Pc reaches a predetermined pressure of 180 mmHg after about 17 seconds from the start of pressurization, and the depressurization process is started from that time point. In the sound signal Ks' of this comparative example, as seen from comparison with the verification result of FIG. 7, a signal corresponding to the Korotkoff sound signal Kc is buried in noises. Therefore, in this comparative example, it can be said to be difficult to extract only the Korotkoff sound from sounds generated by the measurement target site.

Note that in the above-described depressurization process (steps S6 to S9 in FIG. 5), the diaphragm 62 of the Korotkoff sound detection device 60 tends to gradually return to the flat state shown in FIG. 6A from the state of being convexly bent on the side of the other surface 62b as shown in FIG. 6B. When the pressure of the air in the chamber Cm is about to change due to this, the hole 63o for pressure relaxation of the narrow tube 63 serves to communicate air between the inside and the outside of the chamber Cm as indicated by arrow Ai to restrain the pressure of the air in the chamber Cm from changing from the atmospheric pressure Am.

Therefore, not only in the pressurization process (steps S3 and S4 in FIG. 5) but also in the depressurization process (steps S6 to S9 in FIG. 5), the pressure of the air in the chamber Cm is restrained from changing from the atmospheric pressure Am. As a result, it is possible to prevent the sensitivity, resolution, durability, and reliability of the microphone 40 from being adversely affected by the pressure change (load) of the air in the air pipe 37. As the microphone 40, not only a condenser microphone but also various types of microphones such as a dynamic microphone and a micro electronics mechanical system (MEMS) microphone can be adopted, which leading to more freedom in microphone selection.

### Modification

In the above example, the holes 63o, 63oB, and 63oC for pressure relaxation

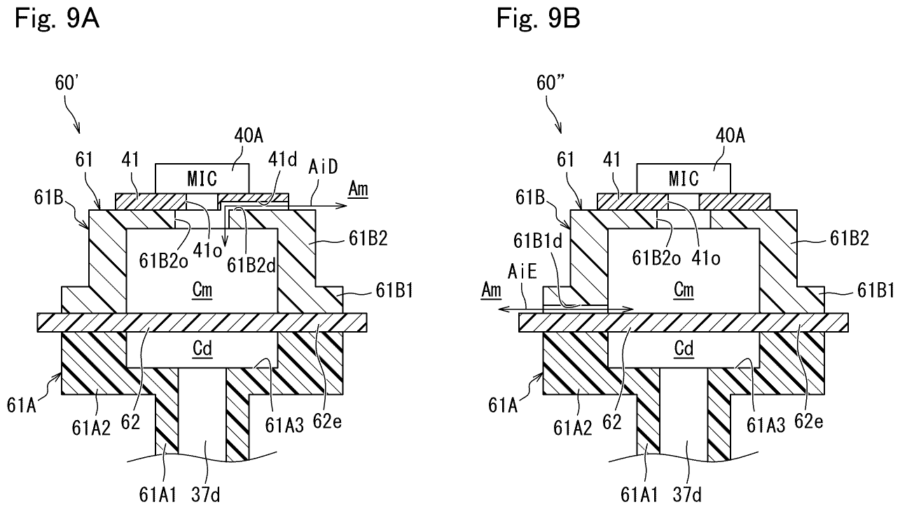

for the chamber Cm are in the form of elongated ducts provided inside the narrow tubes 63, 63B, and 63C, but the present invention is not limited to this. The hole for pressure relaxation may be in a form of, for example, the elongated grooves 41d and 61B2d shown in FIG. 9A or the elongated groove 61B1d shown in FIG. 9B. Note that in FIGS. 9A and 9B, components corresponding to the components previously described are denoted by the same reference signs, and redundant descriptions are omitted.

In the examples of Korotkoff sound detection devices 60′ and 60″ shown in FIGS. 9A and 9B, the cylindrical portion 61B3 of the upper case 61B and the air pipe 38 are omitted from the example of FIG. 2A. Instead, a top portion of the dome portion 61B2 of the upper case 61B is provided with a through hole 61B2o, and a top surface of the dome portion 61B2 is closely attached with a commercially available MEMS microphone 40A having a flat substantially cuboid outer shape via a substrate 41 having a through hole 410. The chamber Cm communicates with the microphone 40A in a fluid flowable manner via the through hole 61B2o of the dome portion 61B2 and the through hole 410 of the substrate 41. Therefore, the microphone 40A can receive, through the air in the chamber Cm, the sound transmitted through the diaphragm 62, convert the sound into the sound signal Ks, which is an electric signal, and output the sound signal Ks. Note that the dimension of the microphone 40A in the planar direction (direction extending flat) is several mm square.

In the example of the Korotkoff sound detection device 60′ shown in FIG. 9A, the hole for pressure relaxation for the chamber Cm includes the elongated groove 61B2d formed on the top surface of the dome portion 61B2 of the upper case 61B and the elongated groove 41d formed at a corresponding position overlapping the elongated groove 61B2d on a lower surface (surface in contact with the dome portion 61B2) of the substrate 41. When the pressure of the air in the air pipe 37 (and the space Cd) gradually changes and the diaphragm 62 bends in the pressurization process or the depressurization process of the cuff 20, and thereby the pressure of the air in the chamber Cm is about to change, these elongated grooves 41d and 61B2d serve to communicate air between the inside and the outside of the chamber Cm through the through hole 61B2o of the dome portion 61B2 as indicated by arrow AiD in FIG. 9A to restrain the pressure of the air in the chamber Cm from changing from the atmospheric pressure Am. Therefore, even if the pressure of the air in the air pipe 37 (and the space Cd) changes, it is possible to prevent the sensitivity, resolution, durability, and reliability of the microphone 40A from being adversely affected by the pressure change (load). Note that one of the elongated grooves 41d and 61B2d may be omitted.

In the example of the Korotkoff sound detection device 60″ shown in FIG. 9B, the hole for pressure relaxation for the chamber Cm includes the elongated groove 61B1d formed in the lower surface (surface in contact with the diaphragm 62) of the plate portion 61B1 of the upper case 61B. When the pressure of the air in the air pipe 37 (and the space Cd) gradually changes and the diaphragm 62 bends in the pressurization process or the depressurization process of the cuff 20, and thereby the pressure of the air in the chamber Cm is about to change, this elongated groove 61B1d serves to communicate air between the inside and the outside of the chamber Cm as indicated by arrow AiE in FIG. 9B to restrain the pressure of the air in the chamber Cm from changing from the atmospheric pressure Am. Therefore, even if the pressure of the air in the air pipe 37 (and the space Cd) changes, it is possible to prevent the sensitivity, resolution, durability, and reliability of the microphone 40A from being adversely affected by the pressure change (load).

Note that a sound insulation material having air permeability and sound insulation properties (e.g., polyurethane foam) may be accommodated in the elongated grooves 41d and 61B2d shown in FIG. 9A and the elongated groove 61B1d shown in FIG. 9B. Thereby, it is possible to prevent the S/N ratio of the Korotkoff sound from decreasing due to the elongated groove 41d, 61B2d, or 61B1d.

Note that the blood pressure measurement by the sphygmomanometer 1 may be performed not in the depressurization process but in the pressurization process.

The measurement target site is not limited to the upper arm, and may be an upper limb other than the upper arm such as a wrist or a lower limb such as an ankle.

As described above, a sphygmomanometer of the present disclosure is a sphygmomanometer that measures a blood pressure based on a Korotkoff sound generated by a measurement target site, the sphygmomanometer comprising:
a cuff for pressure configured to be worn to the measurement target site;
a pump;
an air pipe coupling the cuff and the pump in a fluid flowable manner;
a pressure control unit that supplies and pressurizes a first air to the cuff through the air pipe by the pump in order to press the measurement target site, or discharges and depressurizes the first air from the cuff through the air pipe;
a diaphragm disposed so as to receive, on one surface, a pressure of the first air in the air pipe as a part of a pipe wall of the air pipe or in connection with the pipe wall, the diaphragm being configured to block the pressure of the first air in the air pipe in a pressurization process or a depressurization process of the cuff by the pressure control unit, meanwhile transmit, through this diaphragm, a sound in a frequency band of the Korotkoff sound among sounds traveled through the first air in the air pipe from the measurement target site;
a chamber disposed including the diaphragm as a part of a peripheral wall on a side of an other surface opposite to the one surface with respect to the diaphragm, such that the sound transmitted through the diaphragm travels through a second air contained in the chamber;
a sound detection device provided to face the second air in the chamber in a part other than the diaphragm in the peripheral wall of the chamber, the sound detection device receiving, through the second air in the chamber, the sound transmitted through the diaphragm, and converting the sound into an electric signal; and
a blood pressure calculation unit that calculates the blood pressure of the measurement target site based on the electric signal.

Here, the “one surface” and the “other surface” of the diaphragm refer to both surfaces with a spreading of the diaphragm.

That the diaphragm is disposed “in connection with a pipe wall” of the air pipe includes, for example, a manner of being disposed in connection with a pipe wall of another pipe branched from the air pipe.

In the sphygmomanometer of the present disclosure, in a state where a cuff for pressure is worn to a measurement target site, a pressure control unit supplies and pressurizes a first air to the cuff through the air pipe by the pump in order to press the measurement target site, or discharges and depressurizes the first air from the cuff through the air pipe. In a pressurization process or a depressurization process of the cuff by the pressure control unit, the diaphragm receives, on one surface, a pressure of the first air in the air pipe to block the pressure of the first air in the air pipe. On the other hand, the diaphragm transmits, through this diaphragm, a sound in a frequency band of the Korotkoff sound among sounds traveled through the first air in the air pipe from the measurement target site. As a result, the sound transmitted through the diaphragm travels through a second air in the chamber. The sound detection device receives, through the second air in the chamber, the sound transmitted through the diaphragm, and converts the sound into an electric signal. The blood pressure calculation unit calculates a blood pressure of the measurement target site based on the electric signal.

Here, in this sphygmomanometer, since the diaphragm blocks the pressure of the first air in the air pipe, it is possible to reduce an influence of a pressure pulse wave, which is pulse wave vibration (frequency band; about several tens of Hz) in an artery from sounds generated by a measurement target site. Moreover, the sound detection device receives, through the second air in the chamber, the sound transmitted through the diaphragm, in other words, receives it at a position spaced apart from the diaphragm (directly receives the pressure pulse wave). Therefore, it is possible to further reduce the influence of the pressure pulse wave. Thereby, according to this sphygmomanometer, it is possible to extract a Korotkoff sound with a good S/N ratio from sounds generated by the measurement target site. Therefore, it is possible to enhance an accuracy of blood pressure measurement.

If the chamber is sealed, the diaphragm bends along with a gradual change of the pressure of the first air in the air pipe in the pressurization process or the depressurization process of the cuff, and the pressure of the second air in the chamber changes. If there is such a change in pressure in the second air in the chamber, for example, in a case where the sound detection device is a condenser microphone, a sensitivity of the microphone changes, which is not preferable. Since a width corresponding to a change (about 0 to 300 mmHg) in cuff pressure is required as a dynamic range of the condenser microphone, a resolution of the Korotkoff sound decreases. A durability and a reliability as the sound detection device are also adversely affected.

In the sphygmomanometer of one embodiment,
a part other than the diaphragm in the peripheral wall of the chamber is provided with a hole for pressure relaxation that communicates between an inside and an outside of the chamber in a fluid flowable manner, and the hole for pressure relaxation serves to restrain the pressure of the second air in the chamber from changing from an atmospheric pressure.

In the sphygmomanometer according to this one embodiment, a part other than the diaphragm in the peripheral wall of the chamber is provided with a hole for pressure relaxation that communicates between an inside and an outside of the chamber in a fluid flowable manner. Therefore, when the diaphragm bends along with a gradual change of the pressure of the first air in the air pipe in the pressurization process or the depressurization process of the cuff, and the pressure of the second air in the chamber is about to change, the hole for pressure relaxation serves to restrain the pressure of the second air in the chamber from changing from the atmospheric pressure. Therefore, even if the pressure of the first air in the air pipe changes, it is possible to prevent the sensitivity, resolution, durability, and reliability of the sound detection device from being adversely affected by the pressure change (load).

As the sound detection device, not only a condenser microphone but also various types of microphones such as a dynamic microphone and a micro electronics mechanical system (MEMS) microphone can be adopted, which leading to more freedom in microphone selection.

If the hole for pressure relaxation is a wide opening, noise sounds are likely to enter the chamber from the outside of the chamber through the wide opening. For this reason, there is a possibility that the S/N ratio of the Korotkoff sound decreases.

In the sphygmomanometer of one embodiment,
the hole for pressure relaxation has a form of an elongated duct or an elongated groove.

In the sphygmomanometer according to this one embodiment, the hole for pressure relaxation has a form of an elongated duct or an elongated groove. Therefore, noise sounds are less likely to enter the chamber from the outside of the chamber as compared with a case where the hole for pressure relaxation is, for example, a wide opening. Therefore, it is possible to prevent the S/N ratio of the Korotkoff sound from decreasing due to the hole for pressure relaxation.

In the sphygmomanometer of one embodiment,
a sound insulation material having air permeability and sound insulation properties is accommodated in the elongated duct or the elongated groove.

Here, examples of “sound insulation material having air permeability and sound insulation properties” typically include porous materials such as polyurethane foam.

In the sphygmomanometer according to this one embodiment, the sound insulation material accommodated in the elongated duct or the elongated groove has air permeability. Therefore, the function of the hole for pressure relaxation that prevents the pressure of the second air in the chamber from changing from the atmospheric pressure is not lost due to the presence of the sound insulation material. Since the sound insulation material has sound insulation properties, noise sounds are less likely to enter the chamber from the outside of the chamber through the elongated duct or the elongated groove as compared with a case where only the second air exists in the elongated duct or the elongated groove. Therefore, it is possible to prevent the S/N ratio of the Korotkoff sound from decreasing due to the hole for pressure relaxation.

In the sphygmomanometer of one embodiment,
the diaphragm is set to have a natural frequency matching the frequency band of the Korotkoff sound.

In the sphygmomanometer according to this one embodiment, the diaphragm is set to have a natural frequency matching the frequency band of the Korotkoff sound. Therefore, the diaphragm can selectively transmit a sound in the frequency band of the Korotkoff sound among sounds traveled from the measurement target site through the first air in the air pipe. Therefore, the S/N ratio of the Korotkoff sound can be further improved, and the accuracy of blood pressure measurement can be further enhanced.

In the sphygmomanometer of one embodiment,
the chamber is set to have a resonant frequency matching the frequency band of the Korotkoff sound.

In the sphygmomanometer according to this one embodiment, the chamber is set to have a resonant frequency matching the frequency band of the Korotkoff sound. Therefore, the chamber can selectively amplify the sound in the frequency band of the Korotkoff sound among the sounds transmitted through the diaphragm. Therefore, the S/N ratio of the Korotkoff sound can be further improved, and the accuracy of blood pressure measurement can be further enhanced.

In the sphygmomanometer of one embodiment,
the diaphragm includes a synthetic resin.

In the sphygmomanometer according to this one embodiment, since the diaphragm includes a synthetic resin, the diaphragm is lighter in weight than that including metal, and is easy to process at the manufacturing stage.

The sphygmomanometer of one embodiment further comprises a threshold setting unit that sets a threshold for extracting the Korotkoff sound to the electric signal output by the sound detection device, wherein
the blood pressure calculation unit calculates the blood pressure of the measurement target site based only on a signal exceeding the threshold in the electric signal.

In the sphygmomanometer according to this one embodiment, the threshold setting unit sets a threshold for extracting the Korotkoff sound to the electric signal output by the sound detection device. The blood pressure calculation unit calculates blood pressure of the measurement target site based only on a signal exceeding the threshold in the electric signal. Therefore, for example, background noises can be removed from the electric signal output by the sound detection device. Therefore, the S/N ratio of the Korotkoff sound can be further improved, and the accuracy of blood pressure measurement can be further enhanced.

In another aspect, a blood pressure measurement method of the present disclosure is a blood pressure measurement method for measuring the blood pressure based on the Korotkoff sound generated by the measurement target site by the sphygmomanometer according to claim 1, the blood pressure measurement method comprising:
in the state where the cuff for pressure is worn to the measurement target site,
supplying and pressurizing, by the pressure control unit, the first air to the cuff through the air pipe by the pump in order to press the measurement target site, or discharging and depressurizing the first air from the cuff through the air pipe;
receiving, on the one surface by the diaphragm, in the pressurization process or the depressurization process of the cuff by the pressure control unit, the pressure of the first air in the air pipe to block the pressure of the first air in the air pipe, and transmitting, through this diaphragm, the sound in the frequency band of the Korotkoff sound among sounds traveled through the first air in the air pipe from the measurement target site, such that the sound transmitted through the diaphragm travels through the second air contained in the chamber;
receiving, by the sound detection device, through the second air in the chamber, the sound transmitted through the diaphragm, and converting the sound into the electric signal; and
calculating, by the blood pressure calculation unit, the blood pressure of the measurement target site based on the electric signal.

According to the blood pressure measurement method of the present disclosure, it is possible to extract a Korotkoff sound with a good S/N ratio from sounds generated by a measurement target site, and thus it is possible to enhance an accuracy of blood pressure measurement.

In another aspect, a Korotkoff sound detection device of the present disclosure is a Korotkoff sound detection device that is included in the sphygmomanometer according to claim 1 and extracts a Korotkoff sound from sounds generated by the measurement target site, the Korotkoff sound detection device comprising:
the diaphragm disposed so as to receive, on one surface, the pressure of the first air in the air pipe as the part of the pipe wall of the air pipe or in connection with the pipe wall, the diaphragm being configured to block the pressure of the first air in the air pipe in the pressurization process or the depressurization process of the cuff by the pressure control unit, meanwhile transmit, through this diaphragm, the sound in the frequency band of the Korotkoff sound among sounds traveled through the first air in the air pipe from the measurement target site;
the chamber disposed including the diaphragm as the part of the peripheral wall on the side of the other surface opposite to the one surface with respect to the diaphragm, such that the sound transmitted through the diaphragm travels through the second air contained in the chamber; and
the sound detection device provided to face the second air in the chamber in the part other than the diaphragm in the peripheral wall of the chamber, the sound detection device receiving, through the second air in the chamber, the sound transmitted through the diaphragm, and converting the sound into the electric signal.

According to the Korotkoff sound detection device of the present disclosure, it is possible to extract a Korotkoff sound with a good S/N ratio from sounds generated by a measurement target site.

As clear from the above, according to the sphygmomanometer and the blood pressure measurement method of the present disclosure, it is possible to extract a Korotkoff sound with a good S/N ratio from sounds generated by a measurement target site, and thus it is possible to enhance an accuracy of blood pressure measurement. According to the Korotkoff sound detection device of the present disclosure, it is possible to extract a Korotkoff sound with a good S/N ratio from sounds generated by a measurement target site.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A sphygmomanometer that measures a blood pressure based on a Korotkoff sound generated by a measurement target site, the sphygmomanometer comprising:
a cuff for pressure configured to be worn to the measurement target site;
a pump;
an air pipe coupling the cuff and the pump in a fluid flowable manner;
a pressure control unit that supplies and pressurizes a first air to the cuff through the air pipe by the pump in order to press the measurement target site, or discharges and depressurizes the first air from the cuff through the air pipe;
a diaphragm disposed so as to receive, on one surface, a pressure of the first air in the air pipe as a part of a pipe wall of the air pipe or in connection with the pipe wall, the diaphragm being configured to block the pressure of the first air in the air pipe in a pressurization process or a depressurization process of the cuff by the pressure control unit, meanwhile transmit, through this diaphragm, a sound in a frequency band of the Korotkoff sound among sounds traveled through the first air in the air pipe from the measurement target site;
a chamber disposed including the diaphragm as a part of a peripheral wall on a side of an other surface opposite to the one surface with respect to the diaphragm, such that the sound transmitted through the diaphragm travels through a second air contained in the chamber;
a sound detection device provided to face the second air in the chamber in a part other than the diaphragm in the peripheral wall of the chamber, the sound detection device receiving, through the second air in the chamber, the sound transmitted through the diaphragm, and converting the sound into an electric signal; and
a blood pressure calculation unit that calculates the blood pressure of the measurement target site based on the electric signal.

2. The sphygmomanometer according to claim 1, wherein
a part other than the diaphragm in the peripheral wall of the chamber is provided with a hole for pressure relaxation that communicates between an inside and an outside of the chamber in a fluid flowable manner, and the hole for pressure relaxation serves to restrain the pressure of the second air in the chamber from changing from an atmospheric pressure.

3. The sphygmomanometer according to claim 2, wherein
the hole for pressure relaxation has a form of an elongated duct or an elongated groove.

4. The sphygmomanometer according to claim 3, wherein
a sound insulation material having air permeability and sound insulation properties is accommodated in the elongated duct or the elongated groove.

5. The sphygmomanometer according to claim 1, wherein
the diaphragm is set to have a natural frequency matching the frequency band of the Korotkoff sound.

6. The sphygmomanometer according to claim 1, wherein
the chamber is set to have a resonant frequency matching the frequency band of the Korotkoff sound.

7. The sphygmomanometer according to claim 1, wherein
the diaphragm includes a synthetic resin.

8. The sphygmomanometer according to claim 1, further comprising
a threshold setting unit that sets a threshold for extracting the Korotkoff sound to the electric signal output by the sound detection device, wherein
the blood pressure calculation unit calculates the blood pressure of the measurement target site based only on a signal exceeding the threshold in the electric signal.

9. A blood pressure measurement method for measuring the blood pressure based on the Korotkoff sound generated by the measurement target site by the sphygmomanometer according to claim 1, the blood pressure measurement method comprising:
in the state where the cuff for pressure is worn to the measurement target site,
supplying and pressurizing, by the pressure control unit, the first air to the cuff through the air pipe by the pump in order to press the measurement target site, or discharging and depressurizing the first air from the cuff through the air pipe;
receiving, on the one surface by the diaphragm, in the pressurization process or the depressurization process of the cuff by the pressure control unit, the pressure of the first air in the air pipe to block the pressure of the first air in the air pipe, and transmitting, through this diaphragm, the sound in the frequency band of the Korotkoff sound among sounds traveled through the first air in the air pipe from the measurement target site, such that the sound transmitted through the diaphragm travels through the second air contained in the chamber;
receiving, by the sound detection device, through the second air in the chamber, the sound transmitted through the diaphragm, and converting the sound into the electric signal; and
calculating, by the blood pressure calculation unit, the blood pressure of the measurement target site based on the electric signal.

10. A Korotkoff sound detection device that is included in the sphygmomanometer according to claim 1 and extracts a Korotkoff sound from sounds generated by the measurement target site, the Korotkoff sound detection device comprising:
the diaphragm disposed so as to receive, on one surface, the pressure of the first air in the air pipe as the part of the pipe wall of the air pipe or in connection with the pipe wall, the diaphragm being configured to block the pressure of the first air in the air pipe in the pressurization process or the depressurization process of the cuff by the pressure control unit, meanwhile transmit, through this diaphragm, the sound in the frequency band of the Korotkoff sound among sounds traveled through the first air in the air pipe from the measurement target site;
the chamber disposed including the diaphragm as the part of the peripheral wall on the side of the other surface opposite to the one surface with respect to the diaphragm, such that the sound transmitted through the diaphragm travels through the second air contained in the chamber; and
the sound detection device provided to face the second air in the chamber in the part other than the diaphragm in the peripheral wall of the chamber, the sound detection device receiving, through the second air in the chamber, the sound transmitted through the diaphragm, and converting the sound into the electric signal.
